(19) United States
(12) Patent Application Publication
(10) Pub. No.: US 2026/0069800 A1
(43) Pub. Date: Mar. 12, 2026

(54) MEDICAL DEVICE

(71) Applicant: Drägerwerk AG & Co. KGaA, Lübeck (DE)

(72) Inventor: Markus HAMPE, Lübeck (DE)

(21) Appl. No.: 19/322,902

(22) Filed: Sep. 9, 2025

(30) Foreign Application Priority Data
Sep. 12, 2024 (DE) 10 2024 126 264.8

(51) Int. Cl. A61M 16/00

(52) U.S. Cl. CPC A61M 16/0003; A61M 16/0066; A61M 2209/06

(57) ABSTRACT

A medical device includes a base unit and a cover which can be removed from the base unit and can be opened pivotably with respect to the base unit. The cover can be pivoted for opening optionally about a first axis and/or about a second axis. The second axis is different from the first axis. The cover can be detached from the base unit from the first axis and from the second axis for removal.

### CROSS REFERENCE TO RELATED APPLICATIONS

This application claims the benefit of priority under 35 U.S.C. § 119 of German Application 10 2024 126 264.8, filed Sep. 12, 2024, the entire contents of which are incorporated herein by reference.

### TECHNICAL FIELD

The present invention relates to a medical device comprising a base unit and a cover which can be removed from the base unit and opened pivotably relative to the base unit.

### BACKGROUND

Covers for medical devices can be used to conceal cable and/or tube interfaces and/or other functional elements of the medical device in a closed state and to make them accessible to a user (clinician, service technician) in an open or removed state.

DE 10 2018 005 280 B3 and EP 3 675 942 B1 disclose a ventilator as an example of a medical device that has a base unit with a cover, namely a design cover. The cover can be opened or removed by the user in order to make cable and tube interfaces and other functional elements accessible. DE 10 2018 005 280 B3 and EP 3 675 942 B1 describe three different ways of connecting the cover and the base unit. In a first option, the cover is designed as a door that can be pivoted on one side with a vertical axis. In a second possibility, the cover is designed as a one-sided pivoting door with a horizontal axis arranged near an upper edge. In a third option, the cover can be released and thus removed by unlocking a latching element and sliding it down and thus out of a positive fit formed by tongue and groove elements formed in sections.

A disadvantage of the medical device known from DE 10 2018 005 280 B3 and EP 3 675 942 B1 is that the cover can either be opened pivotably but is not removable or is removable but cannot be opened pivotably. The user's ability to operate the cover in order to access the components behind it is therefore restricted. The accessibility of this medical device is therefore disadvantageously limited.

It is also known from the state of the art to provide a cover designed as a two-part door hinged on both sides. It is also known from the state of the art to provide a two-part cover hinged on one side with the option of convex folding in the middle.

### SUMMARY

It is an object of the present invention to provide a medical device in which a cover can be actuated in a flexible manner.

These and other objects are attained by features according to the invention. The claims, the description and the figures provide advantageous embodiments of the invention.

According to the invention, a medical device is provided, the medical device comprising a base unit and a cover which can be removed from the base unit and opened pivotably relative to the base unit, wherein the cover can be pivoted for opening optionally about a first axis (first pivot axis) and/or about a second axis (second pivot axis), wherein the second axis is different from the first axis, and wherein the cover can be detached from the base unit for removal from the first axis and from the second axis.

In this way, a medical device can be provided with a cover which can be opened by pivoting about a first axis or about a second axis or about the first axis and about the second axis. Additionally, or alternatively and thus optionally, the cover can be removed (detached) from the base unit by taking it off. The user, for example a service technician or a clinician, thus has several needs-oriented options available for accessing components of the medical device located behind the cover, e.g. cable and/or hose interfaces and/or other functional elements, i.e. either by opening or removing the cover, wherein the cover provides both options. When closed, the cover can, for example, conceal an interface area of the base unit. The interface area is thus easily accessible by opening and/or removing the cover, e.g. to connect or remove cables and/or hoses (tubes) in the interface area.

The cover can fulfill various functions, for example some or all of the following: providing a smooth and therefore easy-to-clean surface of the base unit, providing dust protection and mechanical protection of the interface area, providing a desired aesthetic design of the medical device with closed surfaces on all sides of the device.

A cover that can be detached from the first axis and second axis means that the cover can be detached by manual operation, i.e. without tools.

The medical device can, for example, be configured as a ventilator or anesthesia device and has corresponding assemblies and interfaces.

The base unit can be used to accommodate electrical, electronic, mechanical and/or pneumatic components of the medical device. The base unit can be configured as a device chassis or comprise a device chassis and other assemblies, e.g. add-on parts. If the base unit is configured as a device chassis, the cover can, for example, cover one side of the device chassis, such as the rear, when closed. Some or all of the other sides of the device chassis can be covered by other covers of a similar or different design to the cover.

The base unit can be essentially cuboid in shape with a square cross-section or a square layout (footprint), for example.

For example, if the medical device is configured as a ventilator or anesthesia device, it may have a blower for controlling and/or regulating a volume flow and/or a pressure of a gas or gas mixture intended for ventilation. Furthermore, for example, the medical device may have a gas connection for supplying ambient air and/or compressed air and/or oxygen and/or one or more other gases to the medical device. The medical device may further comprise, for example, one or more patient interfaces, such as an inspiratory line and/or an expiratory line. The medical device may further comprise, for example, a control unit and/or an assembly for power supply (e.g. mains (grid power) connection and/or battery and/or accumulator). The medical device may, in particular if it is configured as a ventilator or as an anesthesia device, have further pneumatic components such as lines and valves, e.g. proportional valves, check valves, solenoid valves and the like for influencing the gas flow from the medical device to the patient and from the patient to the environment and/or to the medical device.

The interface area can, for example, have one or more gas inlets and/or outlets, a mains cable, a network cable and/or one or more sensor cables. The interface area can also act as a storage space for excess cable length. A cable winding device can be provided in the interface area for this purpose, for example.

One or more slots for one or more rechargeable batteries (accumulators) may be provided in or on the interface area, e.g. partially or completely coverable by the cover, which may be set up to supply the medical device with electrical energy when it is disconnected from the mains. The cover can protect the slot(s) or the rechargeable battery(s) from access.

The medical device may have a user interface as an integral or connectable component for inputting information by a user and/or for outputting information. For example, the user interface may include a display such as a screen to display information. The user interface can comprise a control unit, for example with input elements such as buttons, keys, rotary switches and/or a display, in particular a touchscreen, for the input of information.

Preferably, the cover is configured in two parts with a first cover part and a second cover part, wherein for a partial opening of the cover the first cover part can optionally be pivoted about a third axis arranged between the first axis and the second axis and the second cover part can be pivoted about the third axis.

In other words, in this preferred embodiment, the cover is not only (fully) openable and removable, but can also be partially opened by optionally pivoting either the first cover part or the second cover part about the third axis in order to expose the area of the base unit (in particular the interface area) located behind the respective cover part. The cover provides both options for partial opening.

In this way, it is possible to (optionally) open the cover only partially, depending on requirements.

Where the term “opening” is used in the context of the invention, this refers to complete opening. Where the term “partial opening” is used in the context of the invention, this refers to partial opening.

Preferably, in order to (fully) open the cover, the first cover part can optionally be pivoted about the third axis in a first step in order to pivot the first cover part in the direction of the second cover part, the second cover part being pivotable about the second axis in a further step in this state (i.e. in the partially opened state). Furthermore, in order to (fully) open the cover, the second cover part can optionally be pivoted about the third axis in a first step in order to pivot the second cover part in the direction of the first cover part, the first cover part being pivotable about the first axis in a further step in this state (i.e. in the partially open state).

The flexibility in handling the cover for opening and partial opening can thus be improved.

In other words, it is preferably possible to first pivot the first cover part in the direction of the second cover part, i.e. to partially open the cover in a first step, and then to pivot both cover parts together about the second axis in order to fully open the cover in a second step. Preferably, the first cover part locks onto the second cover part in the fully pivoted state, thus providing a particularly easy-to-handle unit.

Conversely, it is also possible to first pivot the second cover part in the direction of the first cover part, i.e. to partially open the cover in a first step and then to pivot both cover parts together around the first axis in order to fully open the cover in a second step. Preferably, the second cover part latches onto the first cover part in the fully pivoted state, thus providing a particularly easy-to-handle unit.

Preferably, the first axis is provided by a cover-side first coupling structure and a corresponding base-unit-side first coupling structure, wherein in a connected state of the cover-side first coupling structure and the base-unit-side first coupling structure, it is optionally possible to pivot the cover or only the first part of the cover about the first axis, wherein the cover can be removed by detaching the cover-side first coupling structure from the base-unit-side first coupling structure.

This is a particularly advantageous design of the first axis, as the same structure can be used to provide both the pivoting function for opening and the detachment function for removing. This reduces the complexity of the medical device.

Preferably, the second axis is configured accordingly. In other words, it is preferred that the second axis is provided by a cover-side second coupling structure and a corresponding base-unit-side second coupling structure, wherein in a connected state of the cover-side second coupling structure and the base-unit-side second coupling structure, pivoting of the cover about the second axis is possible, and wherein detachment of the cover-side second coupling structure from the base-unit-side second coupling structure enables removal of the cover.

Preferably, the medical device further comprises a number of latching elements, wherein the number of latching elements is arranged and configured to latch the first cover part and the second cover part together in the partially opened state of the cover.

In this way, the first cover part can latch onto (snap into place on) the second cover part after pivoting about the third axis and either remain secured in this position and thus provide a defined partially open state or be pivoted about the second axis together with the second cover part as an easy-to-handle unit for (complete) opening. The same applies to the second cover part.

The number of latching elements can be a single element or multiple elements (one or a plurality). Each of the latching elements can be configured as a magnet, for example.

Preferably, the first cover part and the second cover part are configured and arranged to rest on each other in the partially open state, in that the third axis is provided by a hinge that opens at an angle of 180°, such as a film hinge.

Preferably, the first coupling structure on the base unit side and the first coupling structure on the cover side are configured and arranged to engage automatically when the first coupling structure on the base unit side is connected to the first coupling structure on the cover side.

In this way, handling can be simplified when inserting the cover into or onto the base unit when the cover is removed from the base unit.

Preferably, the second coupling structure on the base unit side and the second coupling structure on the cover side, if present, are configured and arranged to engage automatically when the second coupling structure on the base unit side is connected to the second coupling structure on the cover side.

Preferably, the automatic engagement is made possible by the first coupling structure on the base unit side and/or the second coupling structure on the base unit side being configured as a number of spring-loaded pins by means of a spring arrangement, so that the number of spring-loaded pins can be displaced in a resetting manner essentially in the direction of the first axis and/or the second axis.

Preferably, the medical device also has a handle element for opening and/or partially opening the cover, wherein the handle element for opening and/or partially opening the cover is movable from a closed state to an open state, wherein the handle element is configured to be self-resetting (to automatically reset to the closed state).

In this way, the handling of the medical device can be improved.

Preferably, the cover has a retaining structure in the area of the third axis, which can be brought into engagement with a retaining contour of the base unit in such a way that when the first cover part and/or the second cover part is partially opened, a positive connection occurs between the retaining structure and the retaining contour, which causes the first cover part and/or the second cover part to latch onto the base unit.

In this way, a defined behavior can be achieved during partial opening and a captive position of the cover can be provided when both cover parts, i.e. both the first cover part and the second cover part, are opened simultaneously.

Preferably, the first cover part and the second cover part can be of essentially the same size, with the third axis being configured and arranged essentially centrally between the first axis and the second axis.

This allows the first cover part and the second cover part to be partially opened without the cover protruding laterally from the base unit.

Preferably, the medical device also has a retainer device (anti-loss device/loss protection) which connects the base unit and the cover to one another, wherein the retainer device is dimensionally stable, and wherein the retainer device has an articulated section, and/or wherein the retainer device has a section which is resiliently deformable (elastically deformable).

If the cover is removed from the base unit, the retainer device can be used to ensure that the cover remains on the device. The dimensionally stable design allows material damping of a jolt if the cover accidentally falls off when removed. The articulated section prevents the retainer device from twisting. The resiliently deformable section improves the function of damping the jolt and thus further reduces the dynamic load on the retainer device and/or a connection point of the retainer device to the base unit if the cover is accidentally dropped.

Preferably, the first axis, the second axis and—if present—the third axis are configured and arranged vertically in the intended state of the medical device. Alternatively, but less preferably, the first axis, the second axis and—if present—the third axis can be configured and arranged horizontally in the intended state of the medical device.

In a particularly preferred combined embodiment, a two-part cover accessible from two sides and preferably divided vertically in the middle is provided, which can initially be opened by approximately 180° from both sides in order to allow easy access to the elements of the interface area configured and arranged on the respective side. In a second step, this design allows the cover, which is folded to approximately 180° and thus doubled, to be opened further, preferably further than 90°, for example by approximately 130°, until the entire interface area is exposed.

These and other features of the invention are also apparent from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a perspective front view of a first embodiment of a medical device according to the invention;

FIG. 1b is a perspective rear view of the first embodiment of the medical device according to the invention as shown in FIG. 1a, showing the cover removed and showing a detail view with the cover removed;

FIG. 2a is a perspective rear view of a second embodiment of a medical device according to the invention, showing the cover closed;

FIG. 2b is a perspective rear view of the second embodiment of the medical device according to the invention as shown in FIG. 2a, showing the cover opened from the left side and pivoted to the right;

FIG. 2c is a perspective rear view of the second embodiment of the medical device according to the invention as shown in FIG. 2a, showing the cover opened from the right side and pivoted to the left;

FIG. 3a is a perspective rear view of a third embodiment of a medical device according to the invention, showing the cover closed;

FIG. 3b is a perspective rear view of the third embodiment of the medical device according to the invention as shown in FIG. 3a, showing the cover partially opened from the left and pivoted about the third axis to the right;

FIG. 3c is a perspective rear view of the third embodiment of the medical device according to the invention as shown in FIG. 3b, showing the cover fully opened and pivoted to the right;

FIG. 3d is a perspective rear view of the third embodiment of the medical device according to the invention as shown in FIG. 3a, showing the cover partially opened from the right and pivoted about the third axis to the left;

FIG. 3e is a perspective rear view of the third embodiment of the medical device according to the invention as shown in FIG. 3d, showing the cover fully opened and pivoted to the left;

FIG. 4a is a perspective rear view of an embodiment of a device cover according to the invention, showing the cover in a closed state;

FIG. 4b is a perspective rear view of the embodiment of the device cover according to the invention as shown in FIG. 4a, showing the cover opened from the left and pivoted about the third axis to the right;

FIG. 5a is a perspective rear view of a fourth embodiment of a medical device according to the invention, showing the cover removed;

FIG. 5b is a perspective rear view of the fourth embodiment of the medical device according to the invention as shown in FIG. 5a, showing the cover opened from the right and pivoted to the left and showing a detail view;

FIG. 6a is a top view of the fourth embodiment of the medical device according to the invention as shown in FIG. 5a, showing the cover removed;

FIG. 6b is in sectional view, taken in the indicated direction along section line A-A according to FIG. 6a, of the fourth embodiment of the medical device according to the invention according to FIG. 5a, shown before closing the cover;

FIG. 7a is a partial top view of the fourth embodiment of the medical device according to the invention as shown in FIG. 5a, showing the cover closed;

FIG. 7b is a sectional view, taken in the indicated direction along section line B-B according to FIG. 7a, of the fourth embodiment of the medical device according to the invention according to FIG. 5a, showing the cover closed;

FIG. 7c is a sectional view, taken in the indicated direction along section line B-B according to FIG. 7a, of the fourth embodiment of the medical device according to the invention according to FIG. 5a, showing the closed cover during an opening process;

FIG. 7d is a sectional view, taken in the indicated direction according to section line C-C according to FIG. 7b, of the fourth embodiment of the medical device according to the invention according to FIG. 5a, showing the cover closed;

FIG. 7e is a sectional view, taken in the indicated direction according to section line C-C according to FIG. 7c, of the fourth embodiment of the medical device according to the invention according to FIG. 5a, showing the closed cover during the opening process;

FIG. 8a is a perspective side view of a fifth embodiment of a medical device according to the invention, shown when opening the cover;

FIG. 8b are perspective views of the fifth embodiment of the medical device according to the invention according to FIG. 8a, showing a handle part and an ejector element with the handle part shown in full (left partial image) and with handle part hidden (right partial image);

FIG. 8c are perspective views of the fifth embodiment of the medical device according to the invention according to FIG. 8a, showing the handle part and ejector element with the handle part shown in full (left partial image) and with handle part hidden (right partial image) and shown during the opening process;

FIG. 9a is a perspective rear view of a sixth embodiment of a medical device according to the invention, showing the second cover part opened by 180° and showing a detail view;

FIG. 9b is a perspective rear view of the sixth embodiment of the medical device according to the invention as shown in FIG. 9a, showing the first cover part open by 130°;

FIG. 9c is a perspective rear view of the sixth embodiment of the medical device according to the invention according to FIG. 9a, showing the first cover part opened by 180° and showing a detail view;

FIG. 9d is a perspective rear view of the sixth embodiment of the medical device according to the invention as shown in FIG. 9a, showing the second cover part open by 130°;

FIG. 10 is a perspective rear view of a seventh embodiment of a medical device according to the invention, showing the cover positioned incorrectly;

FIG. 11 is a perspective rear view of the seventh embodiment of the medical device according to the invention as shown in FIG. 10, showing the cover positioned incorrectly;

FIG. 12a is a perspective rear view of an eighth embodiment of a medical device according to the invention, showing the cover removed;

FIG. 12b is a perspective rear view of the eighth embodiment of the medical device according to the invention as shown in FIG. 12a, showing the cover half open and with a cover pivoted to the right;

FIG. 13a is a perspective rear view of a ninth embodiment of a medical device according to the invention, showing a state in which the first cover part and the second cover part are open at the same time, and enclose an angle of approximately 90°;

FIG. 13b is a perspective rear view of the ninth embodiment of the medical device according to the invention according to FIG. 13a, showing a state in which the first cover part and the second cover part are open at the same time, and enclose an angle of approximately 180°;

FIG. 13c is a sectional view of the ninth embodiment of the medical device according to the invention as shown in FIG. 13a, showing the first cover part and the second cover part enclosing an angle of approximately 90°;

FIG. 13d is a sectional view of the ninth embodiment of the medical device according to the invention as shown in FIG. 13a, showing the first cover part and the second cover part enclosing an angle of approximately 90°;

FIG. 13e is a sectional view of the ninth embodiment of the medical device according to the invention as shown in FIG. 13a, showing the first cover part and the second cover part enclosing an angle of approximately 180°;

FIG. 13f is a sectional view of the ninth embodiment of the medical device according to the invention as shown in FIG. 13a, showing the first cover part and the second cover part enclosing an angle of approximately 180°;

FIG. 14a is an exploded perspective view of an embodiment of a retaining device, showing an isolated representation;

FIG. 14b is a perspective view of the embodiment of the assembled retaining device according to FIG. 14a, showing an isolated representation;

FIG. 14c is a detailed sectional view of the retainer device according to FIG. 14a in the state connected to the cover, showing a closing angle of approximately 90°;

FIG. 14d is a perspective view of a tenth embodiment of a medical device according to the invention, showing a state in which the first cover part and the second cover part are open at the same time and pivoted to the left, with the first cover part and the second cover part at an angle of approximately 130° to the left;

FIG. 14e is a perspective view of the tenth embodiment of the medical device according to the invention according to FIG. 14d, showing a state in which the first cover part and the second cover part are open simultaneously and pivoted to the right, with the first cover part and the second cover part at an angle of approximately 130° to the right;

FIG. 14f is a partial perspective detail view of the tenth embodiment of the medical device according to the invention as shown in FIG. 14d, not showing the cover and showing the retainer device in closed position of the not shown cover;

FIG. 14g is a perspective view of the tenth embodiment of the medical device according to the invention as shown in FIG. 14d, showing the cover hanging down, and showing two detail views;

FIG. 14h is a perspective view of the tenth embodiment of the medical device according to the invention according to FIG. 14d, showing the cover placed on the base unit, and showing a detail view;

FIG. 15 is an exploded front view of an embodiment of a cover according to the invention and showing the retainer device;

FIG. 16a is a rear view of an eleventh embodiment of a medical device according to the invention and showing two detail views;

FIG. 16b is a rear view of the eleventh embodiment of the medical device according to the invention as shown in FIG. 16a, showing the second cover part open, and showing a detail view; and

FIG. 16c is a rear view of the eleventh embodiment of the medical device according to the invention as shown in FIG. 16a, showing the first cover part open, and showing a detail view.

### DESCRIPTION OF PREFERRED EMBODIMENTS

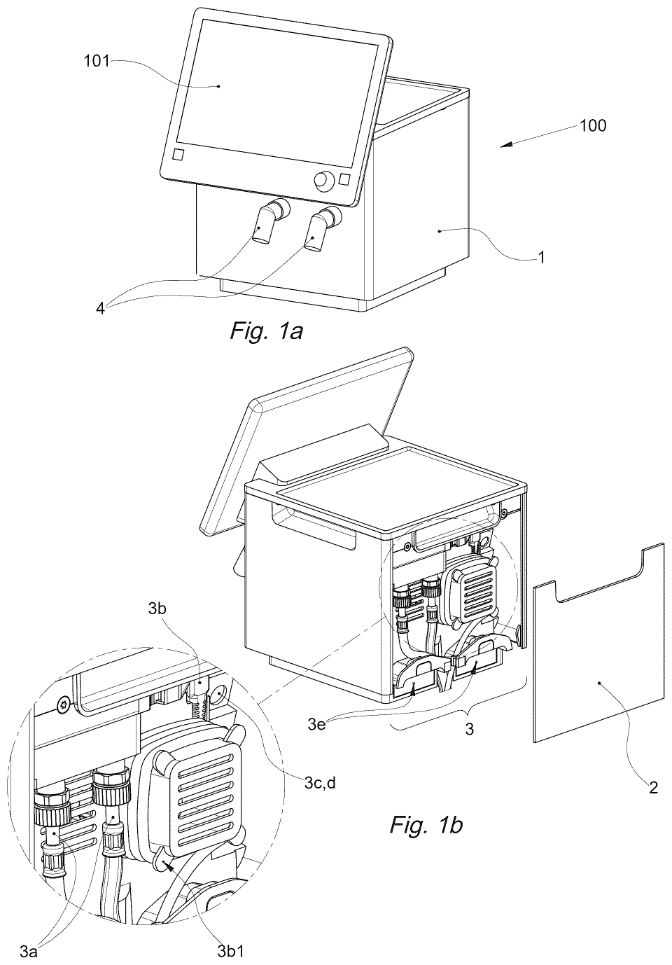

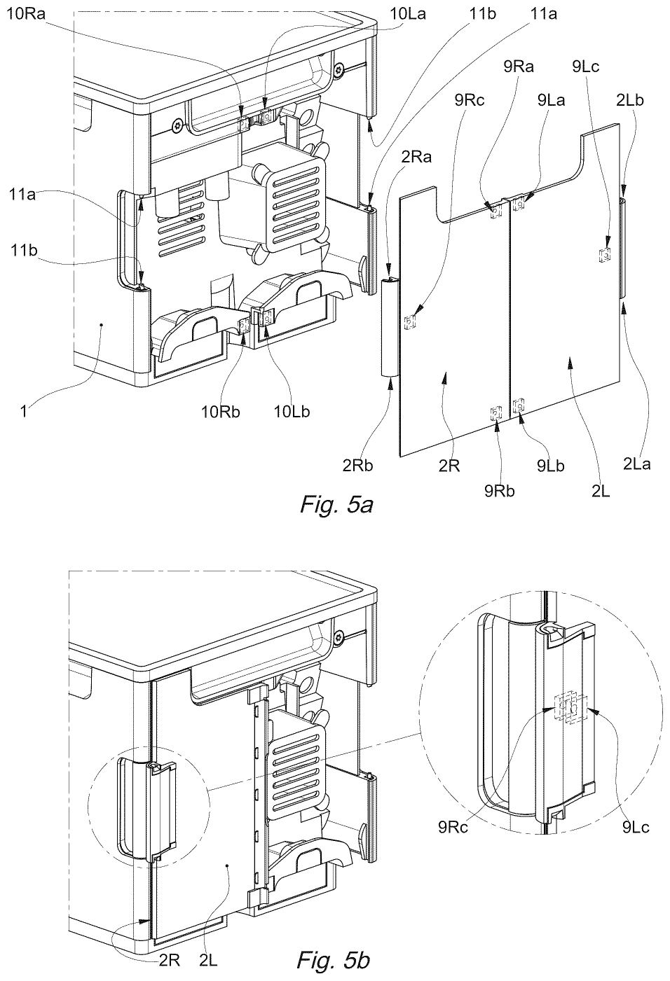

Referring to the drawings, according to the invention, a medical device 100 is provided. Embodiments of medical devices 100 are shown in FIGS. 1a-3e, FIGS. 5a-13f, FIGS. 14c-14h and FIGS. 16a-16c.

If only a medical device 100 or the medical device 100 is referred to in the following, all embodiments and possible embodiments-shown and not shown—are meant equally.

As shown in the embodiments of medical devices 100 according to FIGS. 1a-3e, FIGS. 5a-13f, FIGS. 14c-14h and FIGS. 16a-16c, a medical device 100 is provided which has a base unit 1 and a cover 2, 2L, 2R. The base unit 1 defines an open region covered by the cover 2, 2L, 2R. The cover 2, 2L, 2R can be removed from the base unit 1 and can be opened pivotably with respect to the base unit 1. For opening, the cover 2, 2L, 2R is optionally pivotable about a first axis AL and about a second axis AR, wherein the second axis AR is different from the first axis AL, and wherein the cover 2, 2L, 2R is detachable from the first axis AL and from the second axis AR for removal from the base unit 1.

The base unit 1 is configured to support medical device components. If the medical device 100 is configured as a ventilator or anesthesia device, the base unit 1 can, for example, have an interface 4 for a gas inlet and a gas outlet (see FIG. 1a). Ventilation tubes (hoses) can be connected to the interface 4, which can be led to the patient and from the patient back to the medical device 100. The interface 4 can, for example, be located on the front of the medical device 100. The base unit 1 can also have an interface area 3 (see FIG. 1b), which can, for example, have one or more gas inlets 3a, a power cable connection 3b, a network cable connection 3c and/or one or more sensor cable connections 3d. In order to provide a hygienic and tidy workplace, it is preferred and shown to provide the interface area 3 on one side of the device, in particular the rear.

A space between interface area 3 and cover 2, 2L, 2R can function as a storage space for a mains cable (power cable) that can be connected to the mains cable connection 3b and/or for other cables. A cable winding device 3b1 can be provided for this purpose, for example (see FIG. 1b). One or more slots for rechargeable batteries (accumulators) 3e can also be provided behind the cover 2, 2L, 2R (see FIG. 1b).

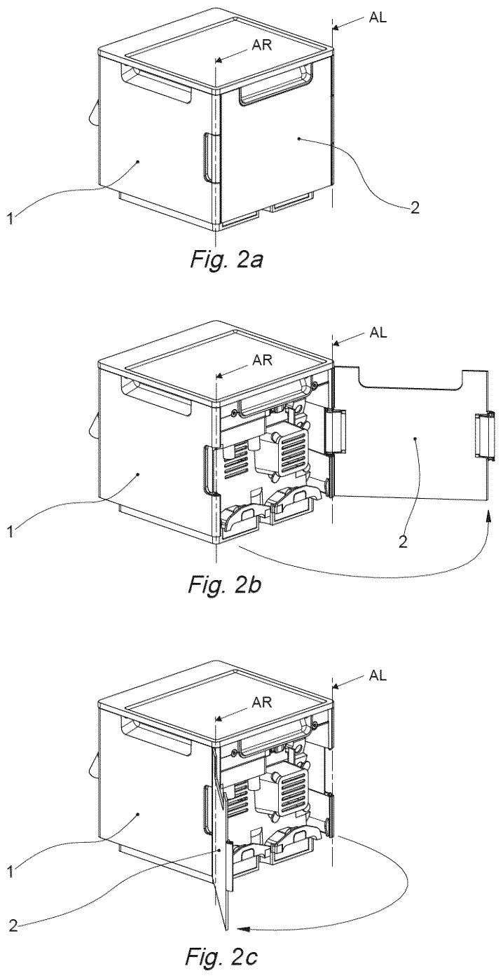

The cover 2, 2L, 2R can be configured in one or more parts. The embodiments according to FIGS. 1a and 1b as well as FIGS. 2a-2c show a one-piece cover in this respect. The other embodiments show a multi-part cover 2L, 2R, which can, for example, be configured in two parts with a first cover part 2L and with a second cover part 2R, wherein for partial opening of the cover 2, 2L, 2R, the first cover part 2L can optionally be pivoted about a third axis B configured and arranged between the first axis AL and the second axis AR and the second cover part 2R can be pivoted about the third axis B. In this case, to open the cover 2, 2L, 2R, optionally the first cover part 2L can be pivotable about the third axis B in order to pivot the first cover part 2L in the direction of the second cover part 2R, the second cover part 2R being pivotable about the second axis AR in this state, and the second cover part 2R can be pivotable about the third axis B in order to pivot the second cover part 2R in the direction of the first cover part 2L, the first cover part 2L being pivotable about the first axis AL in this state.

If the multi-part cover 2L, 2R is configured in two parts, it is preferred that the first cover part 2L and the second cover part 2R are essentially the same size, with the third axis B being configured and arranged essentially centrally between the first axis AL and the second axis AR.

In all embodiments, the first axis AL can be provided by a cover-side first coupling structure 2La, 2Lb and a corresponding base-unit-side first coupling structure 11a, 11b, wherein in a connected state of the cover-side first coupling structure 2La, 2Lb and the base-unit-side first coupling structure 11a, 11b, it is possible to pivot the cover 2, 2L, 2R or only the first cover part 2L about the first axis AL, and it is possible to remove the cover 2, 2L, 2R by releasing the first coupling structure 2La, 2Lb on the cover side from the first coupling structure 11a, 11b on the base unit side.

The second axis AR can be configured accordingly in all embodiments, i.e. provided by a second coupling structure 2Ra, 2Rb on the cover side and a corresponding second coupling structure 11a, 11b on the base unit side, wherein in a connected state of the second coupling structure 2Ra, 2Rb and the second coupling structure 11a, 11b on the base unit side being connected, it is possible to pivot the cover 2, 2L, 2R or only the second cover part 2R about the second axis AR, and it is possible to remove the cover 2, 2L, 2R by releasing the second coupling structure 2Ra, 2Rb on the cover side from the second coupling structure 11a, 11b on the base unit side.

In all embodiments, the first coupling structure 11a, 11b on the base unit side and the first coupling structure 2La, 2Lb on the cover side can be configured and arranged to automatically engage when the first coupling structure 11a, 11b on the base unit side is connected to the first coupling structure 2La, 2Lb on the cover side.

In all embodiments, the second coupling structure 11a, 11b on the base unit side and the second coupling structure 2Ra, 2Rb on the cover side can be configured and arranged to automatically engage when the second coupling structure 11a, 11b on the base unit side is connected to the second coupling structure 2Ra, 2Rb on the cover side.

In all embodiments, it can be seen that the first axis AL and the second axis AR can each be configured as a hinge, which enables a pivoting movement of the cover 2 or the respective cover part 2L, 2R about the respective axis AL, AR on the base unit 1.

In each embodiment, the medical device 100 can have one or more handle elements 7 for opening the cover 2, 2R, 2L, wherein the handle element or elements 7 for opening the cover 2, 2R, 2L are movable from a closed state to an open state, and wherein the handle element or elements 7 are configured to be self-resetting (to the closed state). The cover 2 or the respective cover part 2L, 2R can be opened and optionally removed by means of the or each handle element 7.

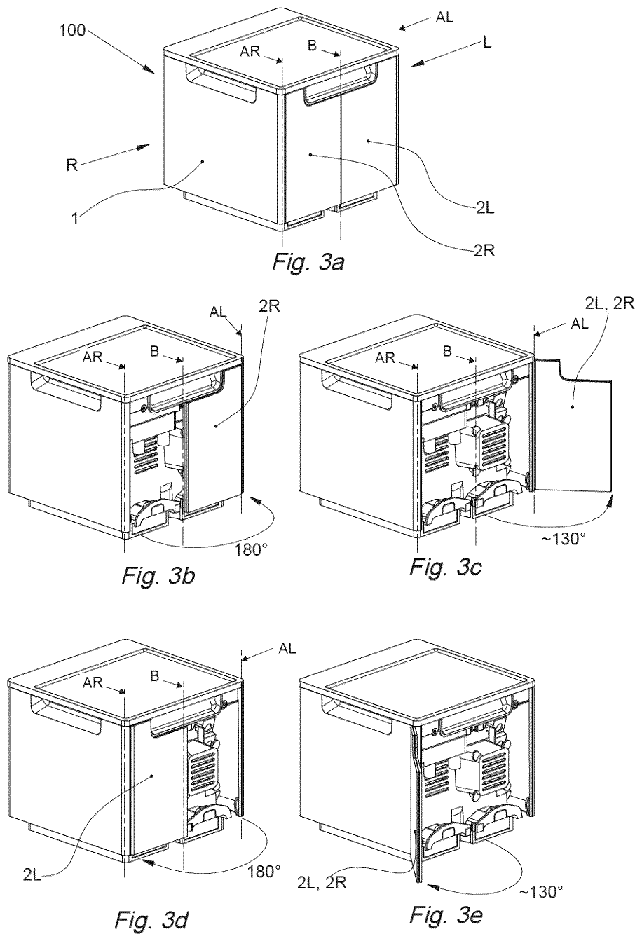

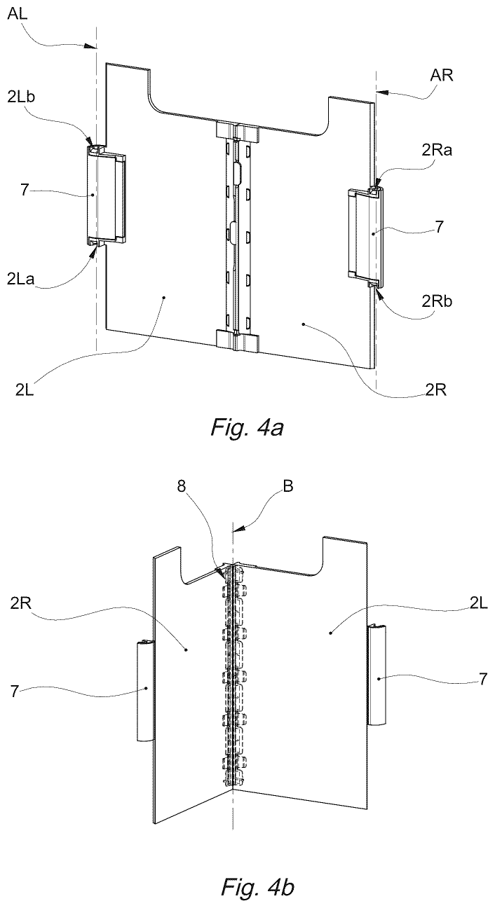

In the embodiments according to FIGS. 3a-13f and 14c-16c, the third axis B can, for example, be provided by a formed central hinge 8, which enables a pivoting movement between the two cover parts 2L, 2R about the third axis B by, for example, 180° in a concave direction. The center hinge 8 can be configured as a film hinge (see FIGS. 4a, 4b). In these embodiments, a two-part cover 2, 2L, 2R, which is directly accessible from both sides and preferably divided vertically in the middle, is thus provided, which can initially be partially opened from both sides by 180° in order to open a double-lying cover 2, 2L, 2R and thus easy access to the elements of the interface area 3 configured and arranged on the respective side, and which can be opened further, namely completely, in a second step, preferably further than 90°, in the embodiments shown in FIGS. 3a-13f and 14e-16c by approximately 130°, until the entire interface area 3 is open.

In all embodiments, the medical device 100 can have a number of latching elements 9La, 9Ra, 9Lc, 9Rc, 9Lb, 9Rb, 10La, 10Ra, 10Lb, 10Rb, wherein the number of latching elements 9La, 9Ra, 9Lc, 9Rc, 9Lb, 9Rb, 10La, 10Ra, 10Lb, 10Rb are configured and arranged to latch the first cover part 2L and the second cover part 2R together in the partially opened state of the cover 2, 2L, 2R. This can be seen in the embodiments according to FIGS. 3a-13f and 14e-16c. For example, some of the latching elements 9Ra, 9La, 9Rb, 9Lb can be provided on the cover 2, 2L, 2R and another part of the latching elements 10Ra, 10La, 10Rb, 10Lb can be provided on the base unit 1. Some or all of the latching elements 9Ra, 9La, 9Rb, 9Lb, 10Ra, 10La, 10Rb, 10Lb can be configured as holding magnets. The latching elements 9La, 9Ra, 9Lc, 9Rc, 9Lb, 9Rb, 10La, 10Ra, 10Lb, 10Rb can be configured and arranged in such a way that they ensure a tight fit of the cover 2, 2L, 2R in the closed and/or partially opened and/or opened state. Latching elements 9Ra, 9La, 9Rb, 9Lb, 10La, 10Ra, 10Lb, 10Rb can also be configured and arranged in such a way that when a cover part 2L or 2R is opened, initially only this cover part 2L or 2R opens and the other cover part 2R or 2L initially remains in the closed position.

Some or all of the latching elements 9Rc, 9Lc can be configured and arranged in such a way that the two cover parts 2L, 2R latch in the 180° or double laid position (see FIGS. 3b, 3d, 5b, 9a, 9c, 12b).

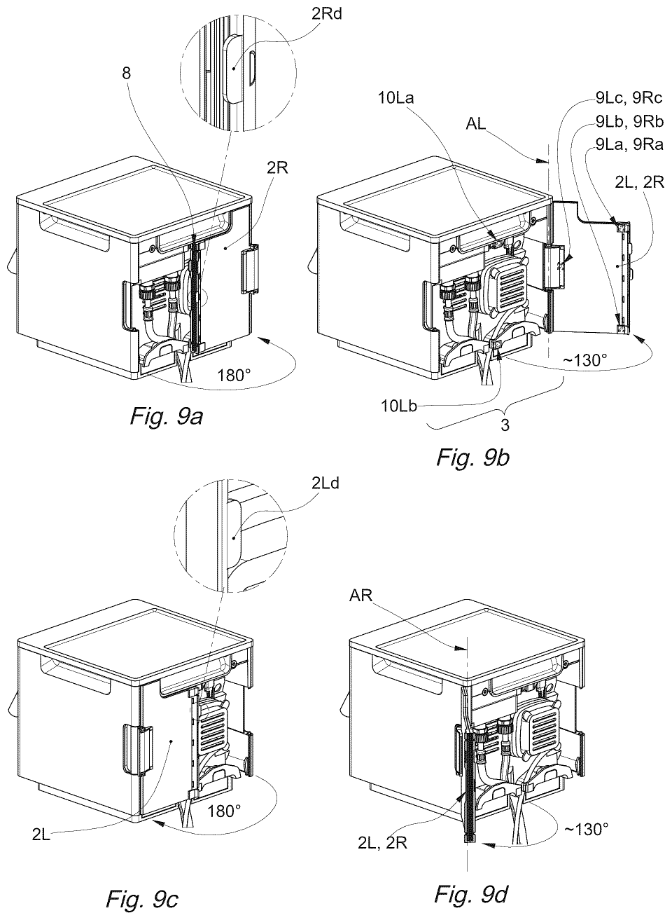

It is possible in all embodiments according to FIGS. 3a-13f and 14c-16c and is particularly evident in FIGS. 9a, 9b, 9c, 9d that, for example, after the second cover part 2R has been opened as described, the second cover part 2R can be moved by 180° against the first cover part 2L with the aid of the center hinge 8 and can be latched to the first cover part 2L by means of the holding magnets 9Lc, 9Rc, so that the side (open region) of the interface area 3 previously covered by the second cover part 2R becomes freely accessible. In this state, it is preferable that the holding magnets 9La, 9Lb of the first cover part 2La, 9Lb still adhere to the holding magnets 10La, 10Lb. If the entire interface area 3 is to be made accessible, in a second step, the composite of first cover part 2L and right cover part 2R can be opened further, e.g. by approximately 130°, about the first axis AL. A grip tab 2Rd formed on the second cover part 2R can be used to actuate the opening process against the force of the holding magnet pairings 9La/10La and 9Lb/10Lb. The same process is also possible from the first side, in which case the first cover part 2L is opened instead as described above, so that initially only the corresponding side of the interface area 3 is accessible. By means of a grip tab 2Ld optionally formed on the first cover part 2L, the composite of the cover parts 2L, 2R can then be opened further, e.g. by approximately 130° about the second axis AR, so that the entire interface area 3 is again accessible.

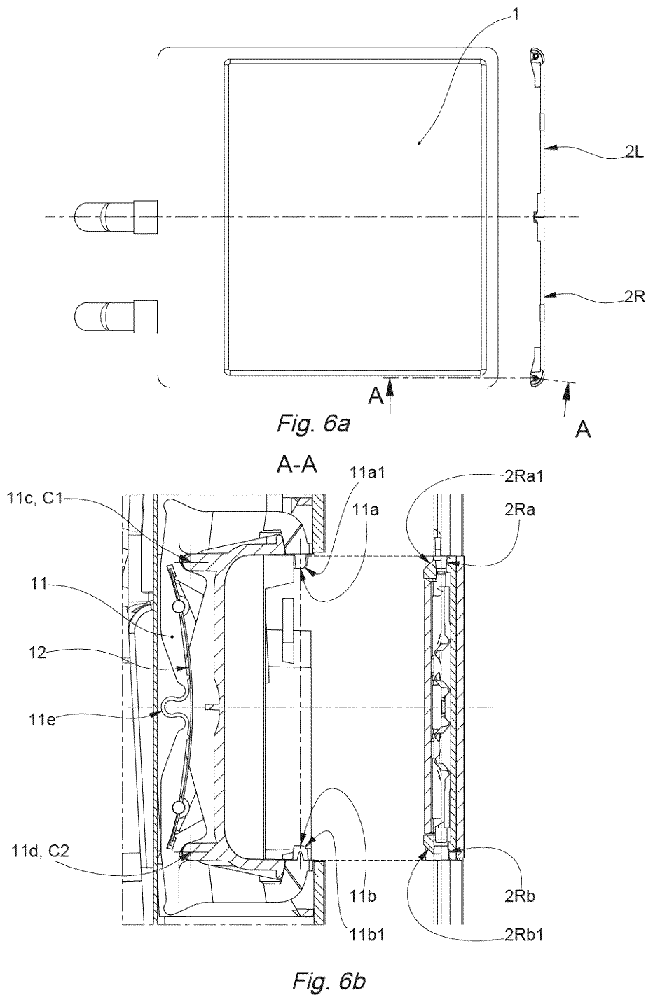

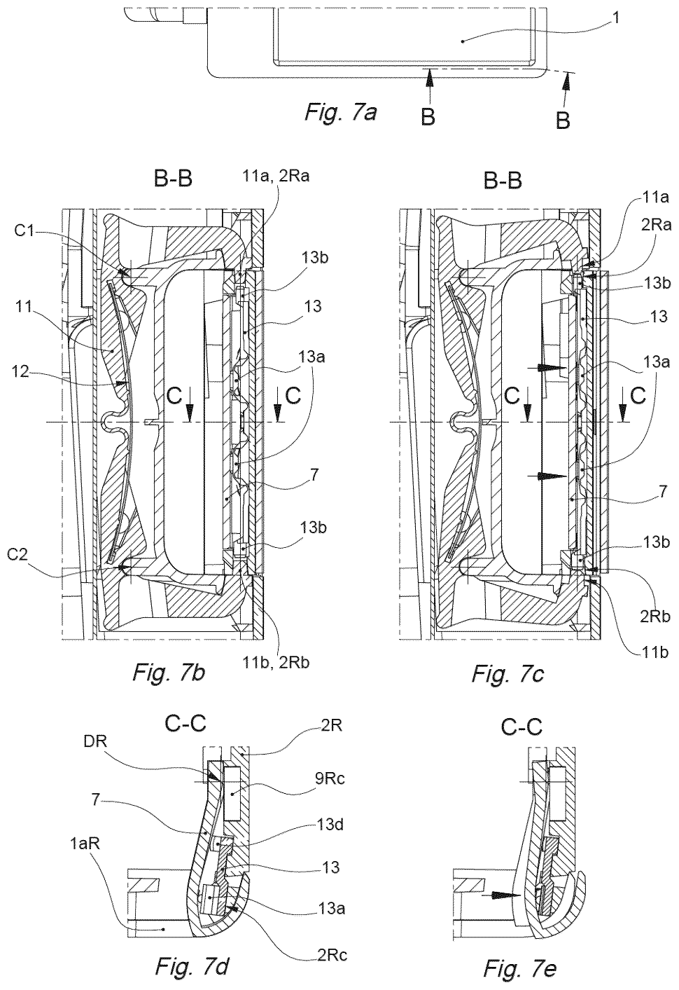

It is possible in all embodiments and is particularly evident in FIGS. 6b, 7b, 7c that the first axis AL and/or the second axis AR can be formed from two concentrically configured and arranged opposing pins 11a, 11b, which can be connected to the base unit 1 and can preferably have a distance of approximately one third of the total height of the cover 2, 2L, 2R from one another. In each embodiment, the cover 2, 2L, 2R can have complementary bores 2Lb, 2La, 2Ra, 2Rb, in which the pins 11a, 11b can engage.

The pins 11a, 11b can be resiliently mounted in the base unit 1 so that they can perform an at least approximately vertical movement against a corresponding spring force. In the embodiments shown, two pins 11a, 11b are each integrated into a component via a latching part (detent part) 11 mounted in the base unit 1, which can perform a rotary movement about an axis of rotation C1, C2 at two bearing points 11c, 11d in the base unit 1. A spring element 12, e.g. a metallic leaf spring 12, supports a spring-back (rebound) effect of a spring 11e configured and arranged centrally on the latching part 11. This mechanism causes the pairs of pins 11a, 11b and bores 2Ra, 2Rb or 2La, 2Lb to engage automatically when the cover 2, 2L, 2R is pressed against the base unit 1. In this case, optional insertion chamfers 11a1, 11a2 can be provided on the pins 11a, 11b and/or in front of the bores 2Ra1, 2Rb1 to simplify engagement. Bores 2Ra, 2Rb, 2La, 2Lb and/or pins 11a, 11b can be conical in order to improve the reliability of engagement and centering and to enable automatic disengagement without damaging components when interference forces act on the cover 2, 2L, 2R.

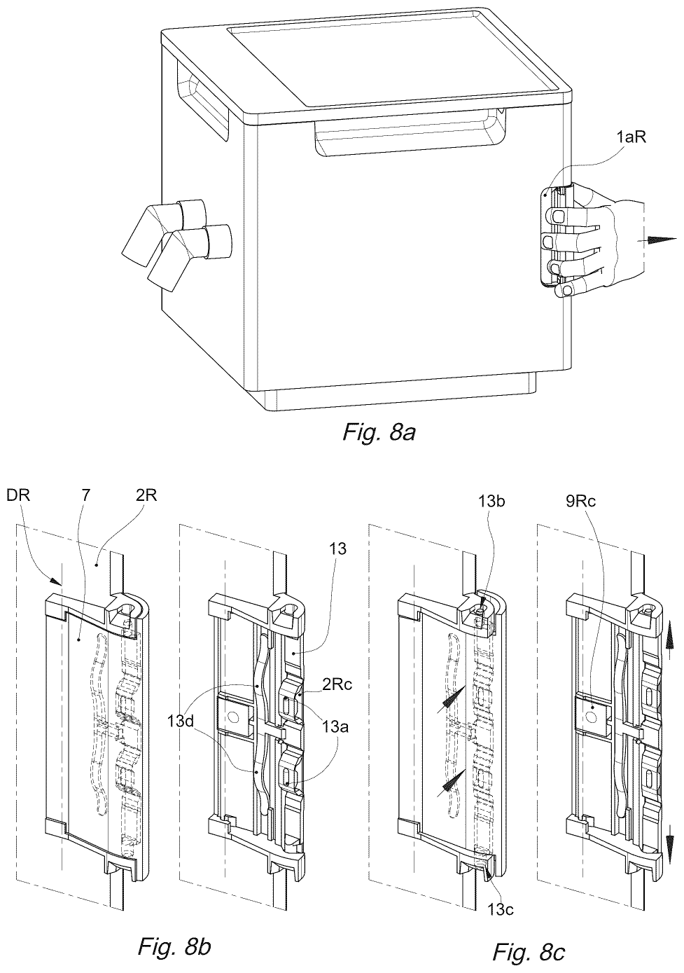

The opening or partial opening of the cover 2, 2L, 2R can be possible in all embodiments, which is particularly evident in FIGS. 7d, 7e, 8a, 8b, 8c, by a user engaging his/her hand in a pocket (recess) laR on the base unit 1 and pulling on a handle part 7 in the opening direction of the cover 2 or the respective cover part 2L, 2R. The corresponding handle part 7 can be mounted on the cover 2, 2L, 2R so that the handle part 7 can rotate about a handle rotation axis DR and consequently rotate by a certain angular amount. As a result, an elastic ejector element 13, which is configured and arranged in a cavity between the inside of the handle part 7 and a sliding surface 2Rc of the cover part 2R, can be compressed and thus increased in length due to a lever geometry 13a, wherein two pins 13b, 13c on the ejector element 13 are pushed through the bores 2Ra, 2Rb and thus push the pins 11a, 11b out of the bores 2Ra, 2Rb until they no longer overlap with them, so that the cover 2 or the respective cover part 2R can be opened.

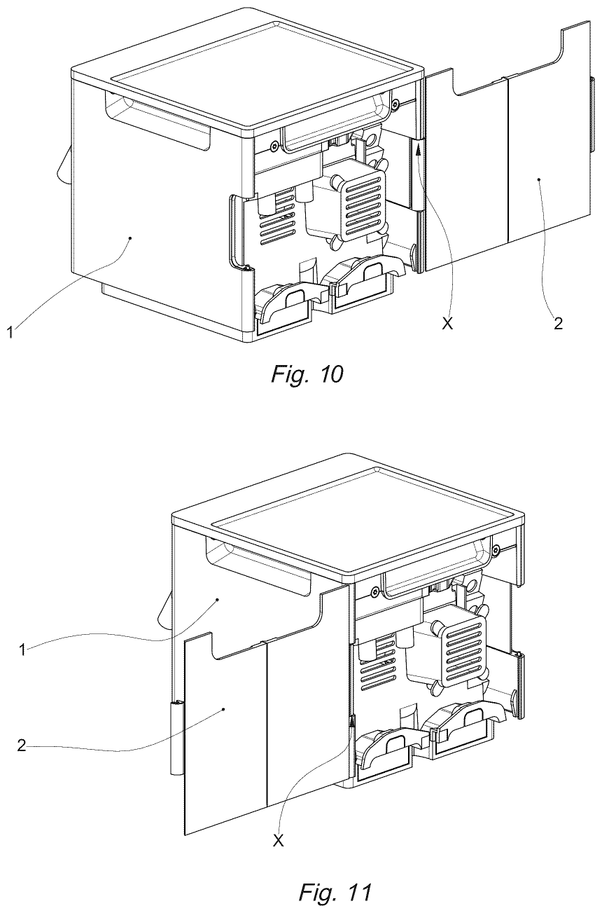

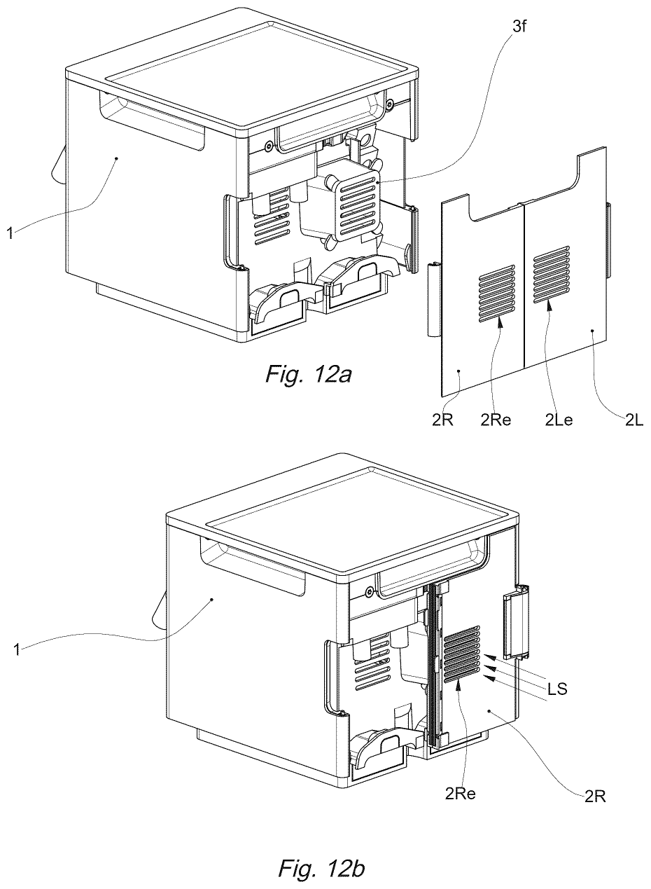

It is possible in all embodiments and particularly evident in FIGS. 10, 11 that it is theoretically possible to incorrectly position the cover 2, 2L, 2R laterally offset on the base unit 1 and still latch it in place, even if the latching geometry is the same on the left and right sides. To avoid this incorrect operation, the pins 11a, 11b and the corresponding bores 2La, 2Lb, 2Ra, 2Rb can be made at least partially different in size. The pairings of pins 11a, 11b and bores 2La, 2Lb, 2Ra, 2Rb can be dimensioned such that the larger pin 11a, 11b does not fit into the smaller bore 2La, 2Lb, 2Ra, 2Rb, so that the engagement only takes place at either the upper or the lower pairing of pin 11a, 11 and bore 2La, 2Lb, 2Ra, 2Rb in the event of incorrect positioning. As a result, the cover 2, 2L, 2R does not latch completely in the event of incorrect positioning and can therefore be easily removed again. In this case, latching is prevented at the points marked X in FIGS. 10, 11 as described.

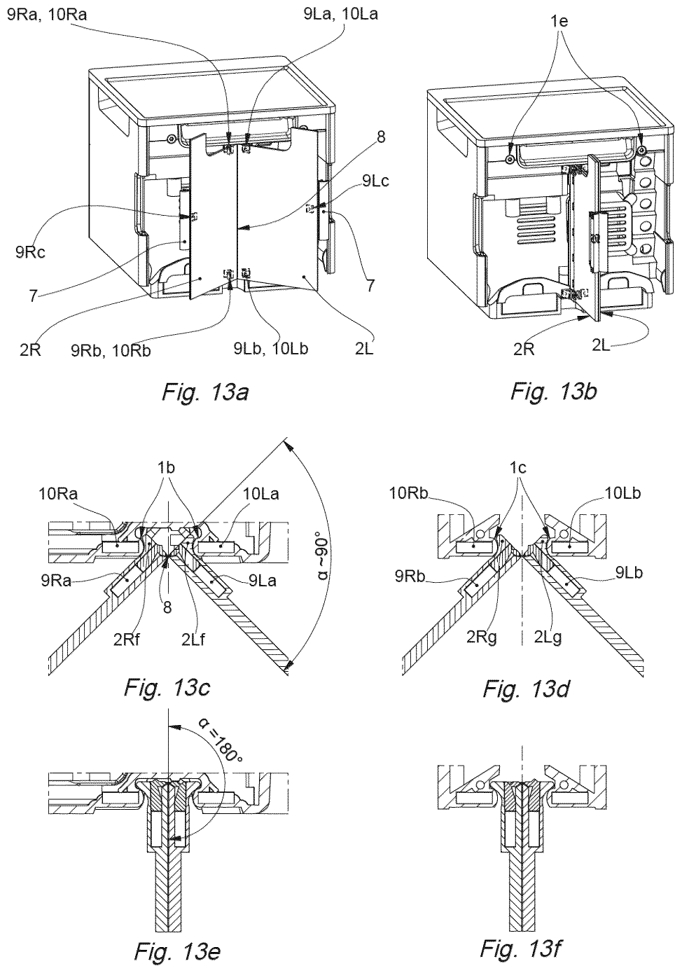

It is possible in all embodiments and is particularly evident in FIGS. 12a, 12b that the cover 2, 2L, 2R can have ventilation openings 2Le, 2Re. If the cover 2, 2L, 2R is configured in two parts, it is preferred that each cover part 2L, 2R has at least one ventilation opening 2Le, 2Re. The ventilation openings 2Le, 2Re can be used to provide a cooling air inlet for a device fan with fan housing 3f that is optionally installed behind it in the interface area 3. It is preferred that similar ventilation openings 2Le, 2Re are provided in a mirror-image arrangement on the two cover parts 2L, 2R, so that in the partially open state, in which both cover parts 2L, 2R lie directly on top of each other, an air flow LS can flow unhindered through both cover parts 2L, 2R.

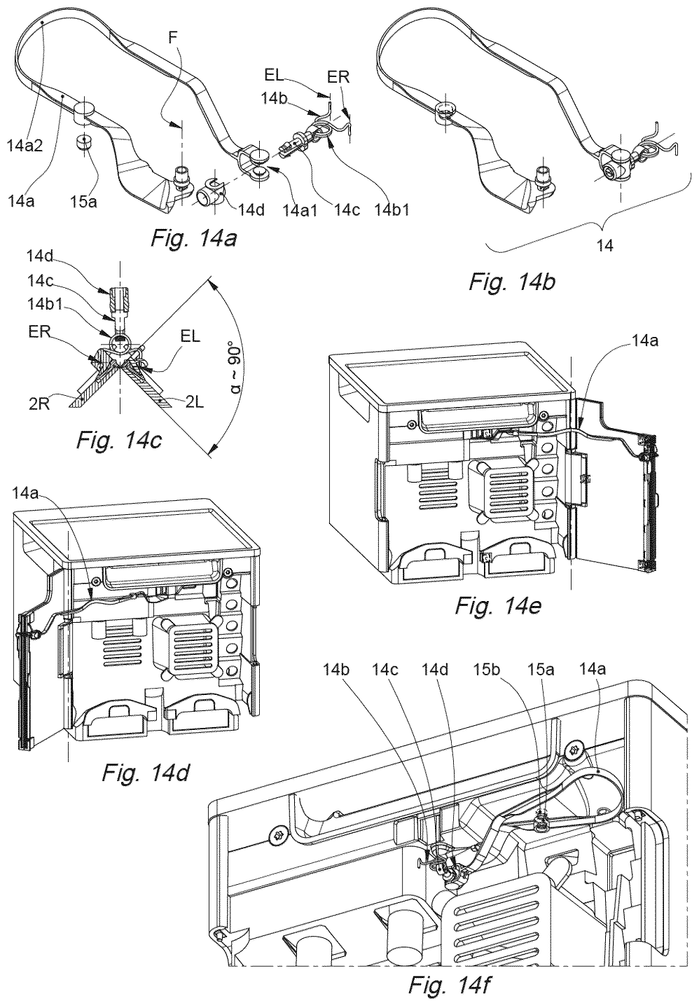

It is possible in all embodiments according to FIGS. 3a-13f and 14c-16c and is particularly evident in FIGS. 13a-13f, that the medical device 100 can be configured in such a way that when the handle part(s) 7 are actuated in order to open both sides simultaneously, the pairs of holding magnets 9La/10La, 9Ra/10Ra, 9Lb/10Lb, 9Rb/10Rb hold back the cover parts 2L, 2R in the central area so that they fold together around the central hinge 8. Initially, only the force of the holding magnet pairs 9La/10La, 9Ra/10Ra, 9Lb/10Lb, 9Rb/10Rb acts, so that the cover parts 2R, 2L can be removed against the magnetic force in a half-open position, as shown in FIG. 13a. In the example shown, this is possible up to a closing angle α of approximately 90° (see FIG. 13c). However, the cover parts 2R, 2L can also remain on the base unit 1 instead and be folded up further. To ensure that they sit firmly on the base unit 1 in the position that can be reached in this way and cannot fall down, a retaining contour 1b on the base unit 1 at the level of the holding magnets 10La, 10Ra (sectional plane of FIG. 13c) and a retaining contour 1c at the level of the holding magnets 10Lb, 10Rb (sectional plane of FIG. 13d) in conjunction with retaining lugs 2Lf, 2Rf, 2Lg, 2Rg, which can be formed on the cover parts 2L, 2R (forming a retaining structure), can be set up for a positive connection from a closing angle α of approximately 90°. Once the center position is reached, the outer holding magnets 9Lc, 9Rc can hold the two cover parts 2L, 2R together, possibly supported by the pairs of holding magnets 9La, 9Ran and 9Lb, 9Rb, so that a stable connection is created. The fixed center position that can be achieved in this way can be useful, for example, to open the base unit 1 for servicing without having to completely remove the cover 2L, 2R from the base unit 1. For example, screw points 1e could be used for this purpose, which are covered when the cover 2L, 2L is closed, but are freely accessible in the center position. Such an assembly concept is described in DE 10 2024 103 650.

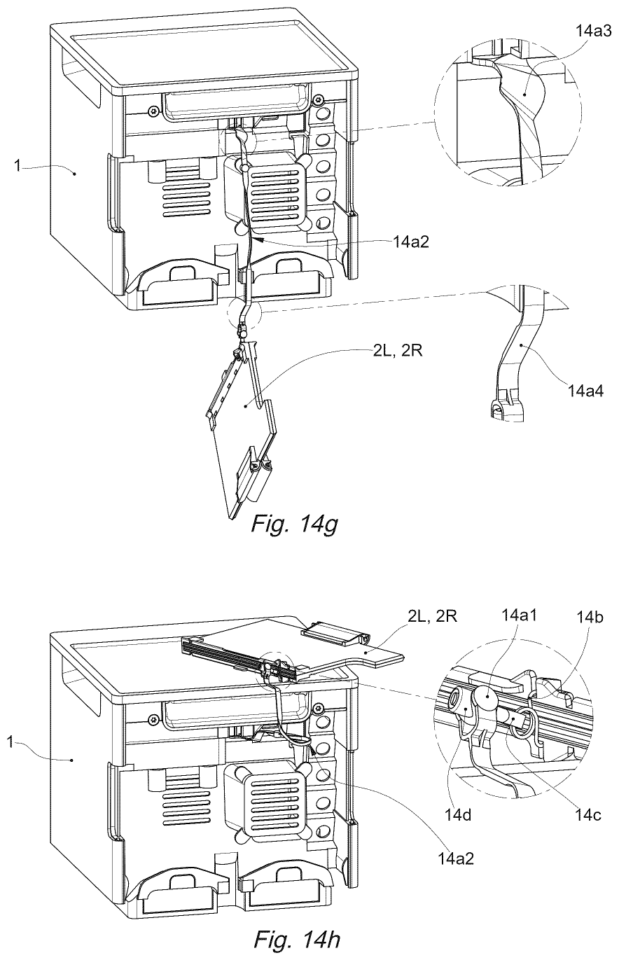

It is possible in all embodiments, and is particularly evident in FIGS. 14a-14c, that the medical device 100 can have an retainer device 14 which connects the base unit 1 and the cover 2, 2L, 2R to one another, wherein the retainer device 14 is dimensionally stable, and wherein the retainer device 14 has a section 14c, 14a1, 14d which is articulated, in particular cardanic, and/or wherein the retainer device 14 has a resiliently deformable section. The retainer device 14 can comprise a U-shaped, pre-formed, semi-rigid retainer strap (catch strap) 14a, which can be made of plastic, for example. The retainer strap 14a can be connected to the cover 2, 2L, 2R via a torsion spring 14b. If the cover 2, 2L, 2R is configured in two parts, it is preferred that the torsion spring 14b is hinged to both cover parts 2L, 2R via a respective spring axis EL, ER, so that the middle area 14b1 of the torsion spring is configured and arranged symmetrically to the cover parts 2L, 2R at any closing angle α.

The articulated section 14c, 14a1, 14d can comprise a cardanic connection. The cardanic connection can comprise an eyelet with bearing pin 14c engaging around the central region 14b1 of the torsion spring 14 and a cardan connector 14d, which can be connected to the torsion spring 14 and thus to the cover 2L, 2R via a fork-shaped articulated connection 14a1.

The retainer strap 14a can be mounted on the base unit 1 with a pivot, which is pivotable about a pivot axis F. When the cover 2L, 2R is opened completely to the left or right, the retainer strap 14a stretches elastically into an approximately straight shape in a preferred embodiment, in that a U-shaped section 14a2 of the retainer strap 14a deforms resiliently. The length of the retainer strap 14a can be dimensioned such that it is fully stretched when the cover 2, 2L, 2R is fully opened, thus preventing further opening. As a further function of the retainer strap 14a, this can prevent the hinge mechanism described above from being overloaded by opening the cover 2, 2L, 2R too far.

The dimensionally stable configuration of the retainer strap 14a means that the retainer strap 14a can move back into a defined position in the interface area 3 when it is closed and therefore cannot come into conflict with cables, hoses or ventilation openings.

Due to its dimensionally stable configuration, the retainer strap 14a can allow the cover 2, 2L, 2R to hang down on the base unit 1. This position can be set intentionally or accidentally if the cover 2, 2L, 2R is dropped during removal. In the latter case, in addition to the U-shaped section 14a2, an optionally inclined-bending section 14a3 and/or a cranked (offset) section 14a4 can be set up to dampen kinetic energy of the cover 2, 2L, 2R as the cover 2, 2L, 2R falls and to influence the direction of fall of the cover 14 in order to reduce a dynamic tensile load.

The dimensionally stable configuration of the retainer strap 14a also allows the cover 2, 2L, 2R to be placed on the top of the medical device 100. A torsional deformation of the U-shaped section 14a2 and the angular mobility between the torsion spring 14b and the eyelet with bearing pin 14c in conjunction with the cardanic connection 14c, 14d, 14al allow almost any position to be selected.

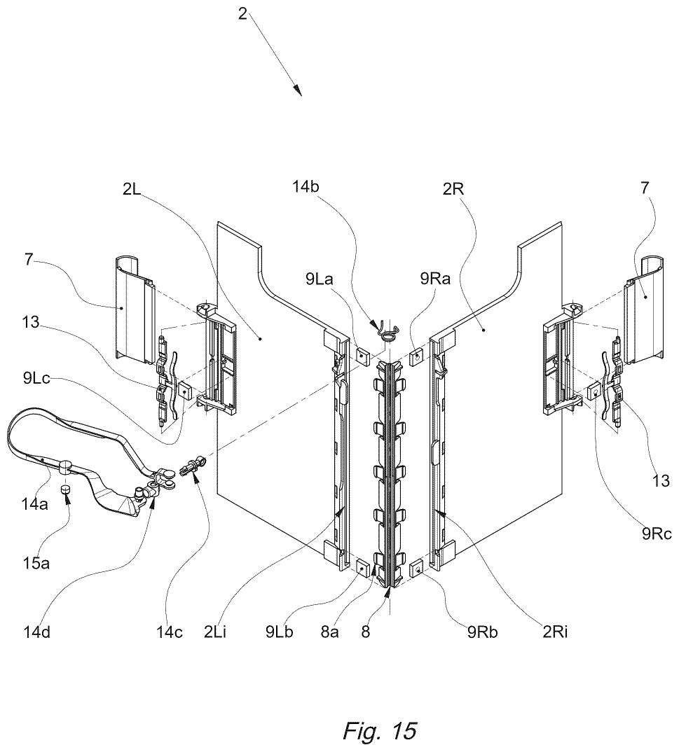

The cover 2, 2L, 2R of each embodiment can be mounted without tools and without additional connecting elements, as can be seen in particular from FIG. 15. The latching elements 9La, 9Ra, 9Lb, 9Rb can be inserted into receptacles, e.g. pockets, of the cover 2, 2L, 2L. The center hinge 8, which is configured as a film hinge, for example, can be inserted and latched onto the cover parts 2L, 2R, for example by means of integrally formed latching hooks 8a in the cover parts 2L, 2R, for example by lateral insertion into corresponding grooves, wherein the latching elements 9La, 9Ra, 9Lb, 9Rb are fixed in the receptacles. The ejector elements 13 can be inserted from the inside through the bores 2Ra1, 2Rb1 or 2La1, 2Lb1 and latched with the cover parts 2L or 2R. The latching elements 9Lc or 9Rc can be inserted into the cover parts 2L, 2R. The handle part or parts 7 can be latched to the cover parts 2L or 2R from the outside, wherein the ejector elements 13 and the latching elements 9Lc or 9Rc can be held, i.e. fixed. The optional torsion spring 14b of the optional retainer device 14 can be bent apart and inserted vertically with the free ends from above or below into corresponding vertical bores on the cover parts 2L, 2R, resulting in mobility about the described spring axes EL and ER. The eyelet with bearing pin 14c can, for example, be divided horizontally in the middle and one half can be threaded through the middle area of the torsion spring 14b1 while bending it slightly open. The cardan connector 14d can be inserted into the fork-shaped joint 14al of the retainer strap 14a and then latched with the eyelet. Finally, an optional holding magnet 15a can be snapped from below into a corresponding receptacle of the retainer strap 14a. This results in a stable connection of all retainer strap parts 14a-14d, 15a.

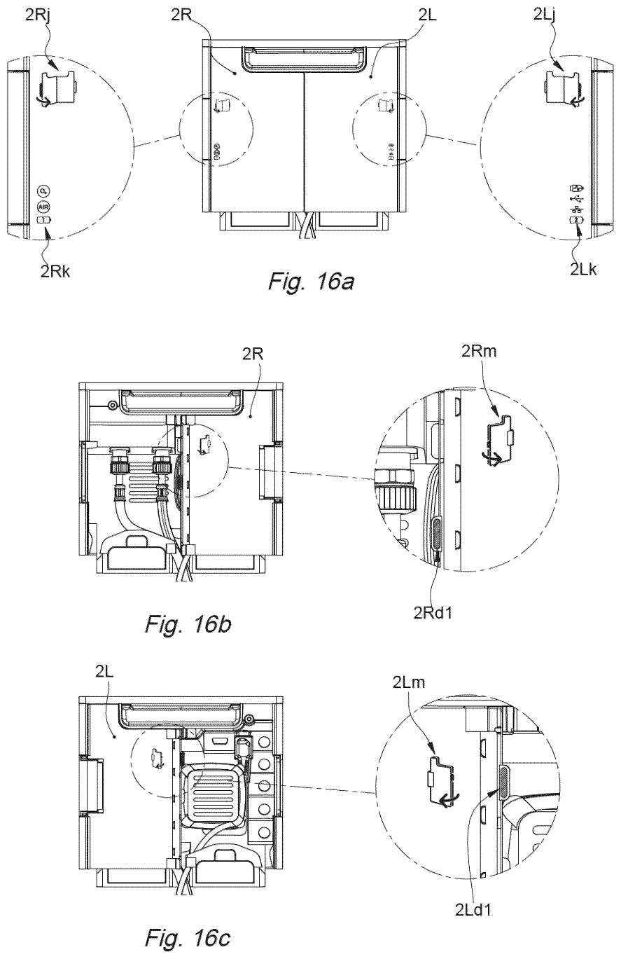

It is possible in all embodiments and shown in FIGS. 16a-16c that the cover 2, 2L, 2R may have help graphics 2Lj, 2Rj, 2Lk, 2Rk, 2Lm, 2Rm, 2Ld1, 2Rd1.

The help graphics 2Lj, 2Rj, 2Lk, 2Rk, 2Lm, 2Rm, 2Ld1, 2Rd1 can be configured and arranged on the outside and/or inside of the cover 2, 2L, 2R and provide information on the actuation of the cover 2, 2L, 2R and/or information on elements of the interface area 3.

In the embodiment according to FIGS. 16a-16c, graphics 2Lj, 2Rj are applied to an outer side (i.e. the side facing outwards in the closed state) of the cover parts 2L, 2R, which illustrate the actuation for opening the first and second cover parts 2L and 2R, respectively. Below this, there may be symbolic representations 2Lk, 2Rk of interface elements that are configured and arranged under the respective cover part 2L, 2R. This makes it possible to see from the outside which cover part 2L, 2R must be opened to access the desired interface element. In the embodiment, sensor connections, USB connection, network connection and a first battery are symbolized in the illustration 2Lk, while O2 and AIR gas connections and a second battery are symbolized in the illustration 2Rk. If a cover part 2L, 2R, e.g. the second 2R, has been opened by 180°, a color marking 2Rd1 on the handle 2Rd, which then becomes visible, and a further representation 2Rm can guide the complete opening process. Corresponding elements 2Ld1, 2Lm can become visible on the first cover part 2L when this is opened accordingly.

All the features of the invention disclosed herein can be combined with one another as desired, provided that this is not contradictory or does not concern alternatives.

While specific embodiments of the invention have been shown and described in detail to illustrate the application of the principles of the invention, it will be understood that the invention may be embodied otherwise without departing from such principles.

### LIST OF REFERENCE SYMBOLS

1 Base unit
1aR Pocket
1b, 1c Retaining contour
1e Screw point
2 Cover
2L First cover part
2R Second cover part
2La, 2Lb First coupling structure on the cover side, bore
2Ra, 2Rb Second coupling structure on the cover side, bore
2Rc Sliding surface
2Ld, 2Rd Grip tab
2Le, 2Re Ventilation opening
2Lj, 2Rj, 2Lk, 2Rk, 2 Lm, 2Rm, 2Ld1, 2Rd1 Help graphic
2Lf, 2Rf Retaining structure
3 Interface area
3a Gas inlet
3b Mains cable (power cable) connection
3b1 Cable winding device 3b1
3c Network cable connection
3d Sensor cable connection
3f Fan housing
4 Interface for gas inlet and gas outlet
7 Handle element
8 Center hinge
8a Latching hook
9La, 9Ra, 9Lc, 9Rc, 9Lb, 9Rb Latching element, holding magnet
10La, 10Ra, 10Lb, 10Rb Latching element, holding magnet
11 Latching part
11a, 11b First coupling structure on the base unit side; second coupling structure on the base unit side; pin
11a1, 11a2 Insertion chamfer
11c, 11d Storage location
11e Spring
12 Spring element, metallic leaf spring
13 Ejector element
13a Lever geometry
13b, 13c Pin of the ejector element
14 Retainer device
14a Retainer strap
14a1 Articulated section of the retainer device, fork-shaped articulated connection
14a2 Resiliently deformable section, u-shaped section
14a3 Resiliently deformable section, inclined-bending section
14a4 Resiliently deformable section, cranked section
14b Torsion spring
14b1 Middle area of the torsion spring
14c Hinged section of the retainer device, eyelet with bearing pin
14d Articulated section of the retainer device, cardan connector
15a Holding magnet
100 Medical device
101 Control unit
α Closing angle
AL First axis
AR Second axis
B Third axis
C1, C2 Rotation axis
DR Handle rotation axis
EL, ER Spring axis
F Trunnion axis
L Left
LS Air flow
R Right

What is claimed is:

1. A medical device comprising:
a base unit; and
a cover, which is configured to be removed from the base unit and opened pivotably relative to the base unit, and wherein the cover is configured to be pivotable about a first axis for opening and is configured to be pivotable about a second axis for opening, wherein the second axis is different from the first axis and wherein the cover is configured to be detachable from the first axis for removal from the base unit and the cover is configured to be detachable from the second axis for removal from the base unit.

2. A medical device according to claim 1, wherein the cover is configured in two parts with a first cover part and with a second cover part, wherein for partial opening of the cover the first cover part is pivotable about a third axis arranged between the first axis and the second axis, and wherein for partial opening of the cover the second cover part is pivotable about the third axis.

3. A medical device according to claim 2,
wherein the cover is configured such that to open the cover the first cover part is pivotable about the third axis to pivot the first cover part towards the second cover part to form a first cover partially open state, wherein the cover is configured such that the second cover is pivotable about the second axis with the first cover part in the first cover partially open state, and
wherein the cover is configured such that to open the cover the second cover part is pivotable about the third axis to pivot the second cover part towards the first cover part to form a second cover partially open state, wherein the cover is configured such that the first cover is pivotable about the first axis with the second cover part in the second cover partially open state.

4. A medical device according to claim 1,
wherein the first axis is provided by a first cover-side coupling structure on a cover side and a corresponding first base-unit-side coupling structure on a base unit side,
wherein in a connected state of the first cover-side coupling structure and the first base-unit-side coupling structure, the cover is pivotable about the first axis, and
wherein the cover is removable by detaching the first cover-side coupling structure on the cover side from the first base-unit-side coupling structure on the base unit side.

5. A medical device according to claim 3, further comprising a number of latching elements, wherein the number of latching elements is configured to latch the first cover part and the second cover part together in the partially opened state of the cover.

6. A medical device according to claim 4, wherein the first base-unit-side coupling structure on the base unit side and the first cover-side coupling structure on the cover side are configured to engage when the first base-unit-side coupling structure is connected to the first cover-side coupling structure.

7. A medical device according to claim 1, further comprising a handle element for opening the cover, wherein the handle element is movable from a closed state to an open state for opening the cover, and wherein the handle element is configured to be self-resetting.

8. A medical device according to claim 2, wherein the cover comprises a retaining structure in a region of the third axis, which is configured to be brought into engagement with a retaining contour of the base unit such that when the first cover part and/or the second cover part is partially opened, a positive connection occurs between the retaining structure and the retaining contour, which positive connection causes the first cover part and/or the second cover part to latch onto the base unit.

9. A medical device according to claim 2, wherein the first cover part and the second cover part are essentially the same size, and wherein the third axis is configured essentially centrally between the first axis and the second axis.

10. A medical device according to claim 1, further comprising a retainer device which connects the base unit and the cover to one another, and wherein the retainer device is dimensionally stable, and wherein the retainer device comprises an articulated section, and/or a resiliently deformable section.

11. A medical device comprising:
a medical device base unit configured to support medical device components, the base unit defining an open region;
a cover, which is configured to cover the open region, to be removed from the base unit and to be opened pivotably relative to the base unit to expose the open region;
a first cover-side coupling structure on the cover and adjacent to a first cover edge;
a first base unit-side coupling structure on the base unit and adjacent to a first edge of the open region, the first cover-side coupling structure and the first base unit-side coupling structure cooperating to form a first axis, wherein in a connected state of the first cover-side coupling structure and the first base unit-side coupling structure, the cover is pivotable about the first axis and the cover is removable by detaching the first cover-side coupling structure from the first base unit-side coupling structure;
a second cover-side coupling structure on the cover and adjacent to a second cover edge;
a second base unit-side coupling structure on the base unit and adjacent to a second edge of the open region, the second cover-side coupling structure and the second base unit-side coupling structure cooperating to form a second axis, wherein in a connected state of the second cover-side coupling structure and the second base unit-side coupling structure, the cover is pivotable about the second axis and the cover is removable by detaching the second cover-side coupling structure from the second base unit-side coupling structure.

12. A medical device according to claim 11, wherein the cover comprises a first cover part and a second cover part and a hinge connection, connecting the first cover part and the second cover part and forming a third axis between the first axis and the second axis, wherein for partial opening of the cover, the first cover part is pivotable about the third axis, and wherein for partial opening of the cover, the second cover part is pivotable about the third axis.

13. A medical device according to claim 12,
wherein the cover is configured such that to open the cover the first cover part is pivotable about the third axis to pivot the first cover part towards the second cover part to form a first cover partially open state, wherein the cover is configured such that the second cover is pivotable about the second axis with the first cover part in the first cover partially open state, and
wherein the cover is configured such that to open the cover the second cover part is pivotable about the third axis to pivot the second cover part towards the first cover part to form a second cover partially open state, wherein the cover is configured such that the first cover is pivotable about the first axis with the second cover part in the second cover partially open state.

14. A medical device according to claim 13, further comprising:
a first cover part latching element; and
a second cover part latching element, wherein the latching elements are arranged to latch the first cover part and the second cover part together in the partially opened state of the cover.

15. A medical device according to claim 12,
wherein the first base-unit-side coupling structure and the first cover-side coupling structure are configured to engage with the first base-unit-side coupling structure connected to the first cover-side coupling structure, and
wherein the second base-unit-side coupling structure and the second cover-side coupling structure are configured to engage with the second base-unit-side coupling structure connected to the second cover-side coupling structure.

16. A medical device according to claim 15, further comprising a first side handle element connected to the first cover part, the first side handle element being movable from a closed state to an open state for disengaging the first base-unit-side coupling structure from the first cover-side coupling structure, wherein the first side handle element is configured to be self-resetting from the open state to the closed state.

17. A medical device according to claim 16, further comprising a second side handle element connected to the second cover part, the second side handle element being movable from a closed state to an open state for disengaging the second base-unit-side coupling structure from the second cover-side coupling structure, wherein the second side handle element is configured to be self-resetting from the open state to the closed state.

18. A medical device according to claim 12, wherein the cover comprises a retaining structure in a region of the third axis, which is configured to be brought into engagement with a retaining contour of the base unit such that with the first cover part and/or the second cover part partially opened, a positive connection occurs between the retaining structure and the retaining contour, which positive connection causes the first cover part and/or the second cover part to latch onto the base unit.

19. A medical device according to claim 12, wherein the first cover part and the second cover part are essentially the same size, and wherein the third axis is arranged essentially centrally between the first axis and the second axis.

20. A medical device according to claim 11, further comprising a retainer device which connects the base unit and the cover to one another, and wherein the retainer device is dimensionally stable, and wherein the retainer device comprises an articulated section, and/or a resiliently deformable section.
